SEQUENCE LISTING (4 sequences)

SEQ ID NO: 1 - protein, 501 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 2 - protein, 456 aa, Homo sapiens
MASMTGGQQMGRGSMAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 3 - DNA, 1467 bp, Homo sapiens
gcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtga

SEQ ID NO: 4 - protein, 488 aa, Homo sapiens
AGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

(19) United States
(12) Patent Application Publication
(10) Pub. No.: US 2009/0214554 A1
(43) Pub. Date: Aug. 27, 2009

(54) TRUNCATED MEMAPSIN 2 COMPOSITIONS AND TREATMENTS

(75) Inventors: Wan Pin CHANG, Edmond, OK (US); Deborah Downs, Oklahoma City, OK (US); Jordan J.N. Tang, Edmond, OK (US)

Correspondence Address: MORRISON & FOERSTER LLP, 755 PAGE MILL RD, PALO ALTO, CA, 94304-1018, US

(73) Assignee: Oklahoma Medical Research Foundation, Oklahoma City, OK (US)

(21) Appl. No.: 12/212,539

(22) Filed: Sep. 17, 2008

Related U.S. Application Data
(63) Continuation of application No. 11/463,501, filed on Aug. 9, 2006, now abandoned.
(60) Provisional application No. 60/707,285, filed on Aug. 10, 2005.

(51) Int. Cl. A61K 39/395; A61K 38/16; A61P 25/28; C07K 16/18; A61K 31/7088

(52) U.S. Cl. 424/139.1; 514/12; 530/387.9; 530/387.3

(57) ABSTRACT

The present invention provides novel methods of reducing memapsin 2 β-secretase activity in a subject, decreasing levels of β-amyloid peptide in the brain of a subject, treating Alzheimer's disease and/or reducing the size and/or number of β-amyloid plaques in the brain of a subject. The methods may include the step of administering an effective amount of truncated memapsin 2 protein, anti-truncated memapsin 2 antibody, and/or nucleic acid encoding a truncated memapsin 2 protein or anti-truncated memapsin 2 antibody. The present invention also provides related pharmaceutical compositions and uses thereof.

### RELATED APPLICATIONS

This application claims benefit of priority to U.S. Provisional Patent Application 60/707,285, filed Aug. 10, 2005, which is hereby incorporated by reference as if fully set forth.

### BACKGROUND OF THE INVENTION

Scientific investigation during the last two decades has substantiated a prominent role for brain amyloid-β(Aβ) in the pathogenesis of Alzheimer's disease. Aβ is a 40- or 42-residue peptide generated from the degradation of β-amyloid precursor protein (APP), a membrane protein, by two proteases known as β-secretase (also known as memapsin 2, the name recommended by IUBMB's Enzyme Nomenclature Commission, or BACE1) and γ-secretase. In this pathway, memapsin 2 initiates the APP cleavage; then the second cleavage by γ-secretase, a multi-protein complex, produces Aβ. An excess level of the neurotoxic Aβ in the brain over a long time leads to the death of neurons, brain inflammation and other harmful events that mark the progression of Alzheimer's disease (AD). At the present, there is no disease-modifying therapy for the clinical treatment of Alzheimer's disease (AD). The few available drugs for treating AD are mostly acetylcholinesterase inhibitors, such as Donepezil (Aricept), which can only mildly improve cognitive performance. Therefore, there is an acute need for the development of new treatments for this disease. Among the potential disease-modifying therapeutic approaches for AD, reduction of Aβ is conceptually the most promising owing to the pivotal role of Aβ in AD pathogenesis. The approach is also supported by experimental results that bring down brain Aβ have been shown to produce benefit in cognitive functions. The importance in this approach is reflected in the active current research for the development and testing of inhibitor drugs targeting to β-secretase, γ-secretase or enzymes in cholesterol synthesis that indirectly affect Aβ production.

Existing evidence supports the contention that Alzheimer's disease (AD) is initiated by an excess level of amyloid-β (Aβ) in the brain. The neurotoxicity of Aβ leads to the death of neurons, inflammation of the brain, dementia and AD (Selkoe, Nature 399A:23-31 (1999); Selkoe and Schenk, Annu Rev Pharmacol Toxicol, 43:545-584 (2003). Since Aβ occupies such a central role in AD pathogenesis, the reduction of Aβ in the brain has become a major therapeutic strategy for AD. Aβ, a 40/42-residue peptide, is derived from the cleavages of amyloid precursor protein (APP) by two proteases known as β-secretase and γ-secretase. Thus, these proteases are major therapeutic targets. The molecular entity of γ-secretase has not yet been conclusively identified although it is clear that this activity is associated with a membrane protein complex consisting presenilin-1, nicastrin and others (Wolfe, Curr. Top. Med. Chem., 4:371-383 (2002). β-Secretase was cloned and identified in our laboratory three years ago as a membrane anchored aspartic protease called memapsin 2 (Lin et al., Proc. Natl. Acad. Sci, USA, 97:1456-1460 (2000). Four other laboratories independently discovered this enzyme and gave different names as BACE (Vassar et al., Science, 286:735-741 (1999) and ASP-2 (Yan et al., Nature, 402:533-537 (1999); Hussain et al., Mol. Cell Neurosci, 14:419-427 (1999). There are, however, several other factors relevant to the Aβ concentration in the brain. Cleavage of APP by α-secretase, which precludes the formation of Aβ, competes for β-secretase cleavage. Cholesterol enhances the endocytosis of β-secretase from cell surface to endosomes, resulting in an increase of Aβ production. The clearance of Aβ from the brain is apparently linked to apolipoprotein E while the degradation of Aβ in the brain are known to be mediated by two peptidases, insulin degradation enzyme (Vekrellis et al., J Neurosci, 20:1657-1665 (2000) and naprilysin (Iwata et al., Nat. Med., 6:143-150 (2000).

Using transgenic AD mice, Aβ immunization was shown to reduce plaques in the brain (Schenk et al., Nature, 400:173-177 (2000) and improve the cognitive functions (Morgan et al., Nature, 408:982-985 (2000); Janus et al., Nature, 408:979-981 (2000). Second-phase clinical trial conducted with 300 patients, however, produced brain inflammation in four patients (Selkoe & Schenk, Annu Rev Pharmacol Toxicol, 43:545-584 (2003) and caused a permanent suspension of the trial. The effectiveness of this therapy requires some of the anti-Aβ antibodies to move across the blood-brain barrier and bind to Aβ-plaques. Plaque removal that follows, therefore, depended on the extent of immune response including the activation of microglial cells (Bard et al., Nature Med, 6:916-919 (2000). Thus, the inflammation of CNS observed in clinical trial was not surprising.

Therefore, there is a need in the art for effective methods of treating Alzheimer's disease. The present invention fulfills this and other needs.

### BRIEF SUMMARY OF THE INVENTION

It has been discovered that administering an effective amount of a truncated memapsin 2 protein, anti-truncated memapsin 2 antibody, nucleic acid encoding a truncated memapsin 2 protein, or nucleic acid encoding an anti-truncated memapsin 2 antibody to a subject provides surprisingly effective reduction of memapsin 2 β-secretase activity, reduction of β-amyloid peptide levels in the brain, reduction of the number and/or size of β-amyloid plaques in the brain, and/or treatment of Alzheimer's disease.

In one aspect, the present invention provides a method of reducing memapsin 2 β-secretase activity to treat a disease associated with memapsin 2 β-secretase activity in a subject in need of such treatment. The method includes administering to said subject an effective amount of a truncated memapsin 2 protein.

In another aspect, the present invention provides a method of decreasing levels of β-amyloid peptide in the brain of a subject. The method includes administering to said subject an effective amount of a truncated memapsin 2 protein.

In another aspect, the present invention provides a method of reducing the size or number of β-amyloid plaques in the brain of said subject. The method includes administering to said subject an effective amount of a truncated memapsin 2 protein.

In another aspect, the present invention provides a method of treating Alzheimer's disease in a patient in need of such treatment. The method includes administering to said subject an effective amount of a truncated memapsin 2 protein.

In another aspect, the present invention provides an antibody specifically immunoreactive with a truncated memapsin 2 protein.

In another aspect, the present invention provides a pharmaceutical composition including a truncated memapsin 2 and a pharmaceutically acceptable adjuvant.

In another aspect, the present invention provides a pharmaceutical composition including a nucleic acid encoding a truncated memapsin 2 and a pharmaceutically acceptable excipient.

### BRIEF DESCRIPTION OF THE DRAWINGS

FIG. 1. A schematic presentation of regions of pre-pro-memapsin 2.

FIG. 2. Plasma Aβ₄₀ and titer of anti-memapsin 2 antibodies in the first cohort of AD mice Tg2576 immunized with promemapsin 2 and truncated memapsin 2. In period 1, the experimental mice were immunized with 6 μg of truncated memapsin 2 per animal. In period 2, each mouse was immunized with 100 μg of promemapsin 2. In period 3, each mouse was immunized with 6 μg of truncated memapsin 2. Asterisks denote statistically significant (p<0.05) differences between experimental and control data. The designation hu Memapsin2 in the legend indicates animals immunized with either promemapsin 2 or truncated memapsin 2.

FIG. 3. Plasma Aβ₄₀ in AD mice Tg2576 immunized with promemapsin 2. The second cohort of experimental mice were immunized with 6 μg of promemapsin 2 per animal. No significant differences were found between experimental (proM2) and controls (immunized with PBS).

FIG. 4. Human prepromemapsin 2. Solid underlined sequence (amino acids 1-13) is a portion of the signal peptide excluded from the recombinant promemapsin 2 construct (FIG. 5). Dotted line appears below sequence of the transmembrane domain; dashed line appears below sequence of the cytosolic domain (including Lys⁵⁰¹).

FIG. 5. illustrates a Human promemapsin 2. Solid underlined amino acids 1-15 are vector-derived amino acids from the T7 promoter of the pET11a expression plasmid. Dashed underlining denotes the activation peptide region, with the homologous amino terminus of pepsin at amino acid Glu⁶⁴ denoted with a dot. Single arrows indicate cleavage positions of clostripain, and double arrow indicates position of spontaneously activated memapsin 2. Both methods were used to create truncated memapsin 2 preparations used for immunizations.

FIG. 6. SDS-PAGE of promemapsin 2 and truncated memapsin 2 preparations used as immunogens. A, promemapsin 2. Lane 1, promemapsin 2 standard (mobility designated proM2), lane 2, truncated memapsin 2 (faint band), lanes 3-5, promemapsin 2 preparations used for immunization of animals. B, truncated memapsin 2. Lane 1, molecular weight standards (kDa), lane 2, promemapsin 2 standard, lane 3, truncated memapsin 2 standard, lane 4 and 5, truncated memapsin 2 preparations used for immunization of animals.

FIG. 7. Nucleic Acid Sequence of one embodiment of a truncated memapsin 2 protein.

FIG. 8. Effect of immunization with attenuated memapsin 2 on plasma Aβ₄₀ concentration (upper panel) and memapsin 2 antibody titer (lower panel). Arrows in lower panel indicate dates of immunization with either PBS (PBS group) or with 6 μg of attenuated memapsin 2 in 100 μl (Mep 2 group). Titers of memapsin 2 antibody in the plasma (lower panel) are shown as solid circles for the immunized group and open circles for the control group (at baseline). In both panels, the * symbol indicates a statistically significant difference where p<0.05; the ̂ symbol marks the data where p<0.1 in comparison with controls.

FIG. 9. Cognitive improvement in reference memory training and spatial memory retention seen for animals immunized with attenuated memapsin 2 (Mep 2 ) or PBS. A, Reference memory training with hidden platform. B, Day 3 probe trial annulus crossing index (ACI). C, Quadrant occupancy from probe trial. Test platform location in SW quadrant; NE quadrant is opposite.

FIG. 10. Effect of attenuated memapsin 2 immunization on the amyloid load and Aβ levels in the brains of 15-month old Tg2576 mice. A. Amyloid plaque number (upper panel) and total plaque area (lower panel) of adjuvant emulsified PBS control (n=13) and attenuated memapsin 2 (Mep 2) immunized (n=11) mouse brain stained with anti-Aβ MAB1561 antibody (Chemicon). Both Aβ plaque numbers and occupied areas are markedly decreased in brains with attenuated memapsin 2 immunization in both areas (* p<0.05; ̂ p<0.15). B. Amounts of Aβ₄₀ and Aβ₄₂ from CHAPS- and guanidine-buffer extracts of mouse brains. Asterisks indicate p<0.05 (n=13 or 11 for control or Mep 2 groups, respectively). C. Western blot of Aβ₄₀ and Aβ₄₂ extracted with guanidine-buffer from brain of attenuated memapsin 2-immunized and control (PBS) mice.

### DETAILED DESCRIPTION OF THE INVENTION

### Definitions

“Nucleic acid” refers to deoxyribonucleotides or ribonucleotides and polymers thereof in single- or double-stranded form, or complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleic acids also include complementary nucleic acids.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically about 18 to 350 amino acids long, e.g., the transmembrane regions, pore loop domain, and the C-terminal tail domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An exemplary algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2× SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

“Polypeptide” refers to a polymer in which the monomers are amino acids and are joined together through amide bonds, alternatively referred to as a “protein.” The terms “protein” encompasses polypeptides. Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the various immunoglobulin diversity/joining/variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)), having antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al (1994) J Immunol: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain of an antibody, including the heavy chain of an Fv, scFv, or Fab. References to “V_{L}” or a “VL” refer to the variable region of an immunoglobulin light chain, including the light chain of an Fv, scFv , dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The radioisotope may be, for example, ³H, ¹⁴C, ³²P, ³⁵S, or ¹²⁵I. In some cases, particularly using antibodies against the proteins of the invention, the radioisotopes are used as toxic moieties, as described below. The labels may be incorporated into the GPR64 nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). The lifetime of radiolabeled peptides or radiolabeled antibody compositions may extended by the addition of substances that stabilize the radiolabeled peptide or antibody and protect it from degradation. Any substance or combination of substances that stabilize the radiolabeled peptide or antibody may be used including those substances disclosed in U.S. Pat. No. 5,961,955.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background.

Specific binding to an antibody under such conditions requires an antibody that is selected for its specificity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with truncated memapsin 2 protein and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988)).

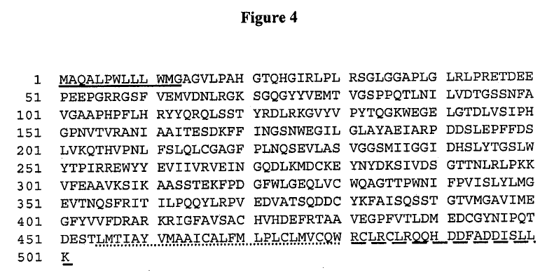

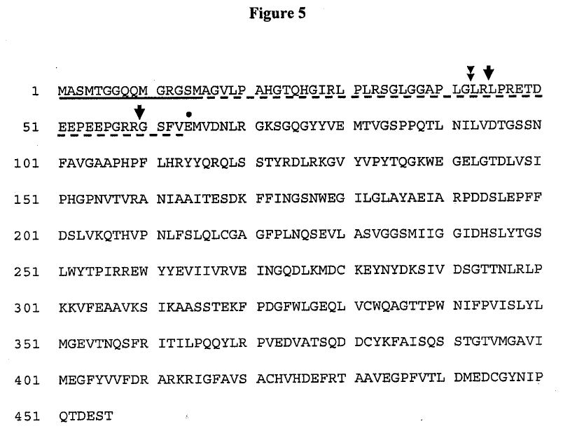

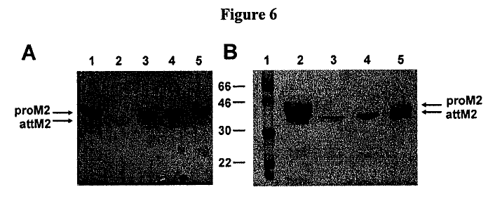

The term “consisting essentially of,” as used herein in reference to a truncated memapsin 2 protein, excludes any portions of a memapsin 2 protein N-terminal to the recited sequence. For example, where the truncated memapsin 2 protein consists essentially of an amino acid sequence with at least 80% sequence identity to an amino acid sequence of amino acids 15-501 of FIG. 4, the truncated memapsin 2 protein does not include amino acids 1-14 of FIG. 4.

### I. Introduction

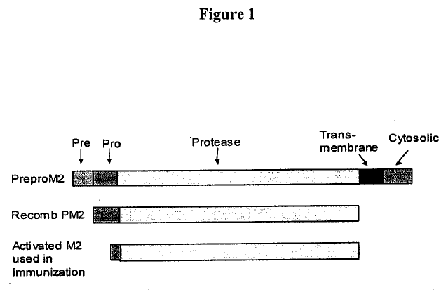

Memapsin 2 β-secretase (also referred to as Memapsin 2, β-secretase, and/or BACE1) is a class I membrane protein that includes a protease domain highly homologous to pepsin, a transmembrane domain and a cytosolic domain (FIG. 1). The protease is synthesized in vivo with an N-terminal pro-region, which is cleaved by furin to remove a 33-residue pro-segment en route to the cell surface (Capell et al., J. Biol. Chem., 275:30849-30854 (2000). The crystal structure of memapsin 2 protease domain (Hong et al., Science, 290:150-1532000) shows that it contains an extended active-site cleft characteristic of aspartic proteases. A β-hairpin flap covers over the active-site cleft. As in other aspartic proteases, the flap must open to permit the entering of substrate into the active-site cleft.

Memapsin 2 initiates cleavage of amyloid precursor protein (APP) leading to the production of β-amyloid (Aβ) and the onset of Alzheimer's disease (AD). It is a major therapeutic target for the development of treatment for AD. The native APP is a poor substrate of memapsin 2 (Lin et al., Proc. Natl. Acad. Sci, USA, 97:1456-1460 (2000); Ermolieff et al., Biochemistry, 39(40):12450-12456 (2000). Swedish mutation at P₂-P₁ subsites from Lys-Met to Asn-Leu enhances the hydrolytic efficiency by about 60 fold, increases Aβ production and manifests an early onset form of AD. The specificity of all eight substrate residues has been determined (Turner et al., Biochemistry, 40:10001-10006 (2001). Native memapsin 2 is glycosylated by three N-linked oligosaccharides. The hinge which links the catalytic unit to the transmembrane region is only 6 residues (Hong et al., Science, 290:150-153 (2000). The transmembrane domain contains three cysteines which are covalently linked to palmitic acids. This is consistent with the lipid raft localization of memapsin 2 in the membranes. The intracellular domain contains a signal for endocytosis from cell surface to endosomes, which likely involve the recognition of proteins such as GGA for transport through the clathrin-coated vesicles (He et al., FEBS Letters, 524:183-187 (2002). The optimal pH for memapsin 2 activity is about 4.5.

### II. Truncated Memapsin 2 Proteins, Anti-Truncated Memapsin 2 Antibodies, and Encoding Nucleic Acids

A. Truncated Memapsin 2 Protein

In some embodiments, the present invention provides a truncated memapsin 2 protein useful in reducing memapsin 2 β-secretase activity in a subject, decreasing levels of β-amyloid peptide in the brain of a subject, treating Alzheimer's disease and/or reducing the size and/or number of β-amyloid plaques in the brain of a subject.

An “truncated memapsin 2 protein,” as used herein, refers to a memapsin 2 protein in which at least a portion of the memapsin 2 N-terminal pro-region is not present. The memapsin 2 N-terminal pro-region is defined herein as that portion of a memapsin 2 protein that is cleaved by furin or a furin-like convertase.

Furin is a ubiquitous subtilisin-like proprotein convertase. It is the major processing enzyme of the secretory pathway and is localized in the trans-golgi network (van den Ouweland, A. M. W. et al. (1990) Nucl. Acids Res., 18, 664; Steiner, D. F. (1998) Curr. Opin. Chem. Biol., 2, 31-39). Substrates of Furin include blood clotting factors, serum proteins and growth factor receptors such as the insulin-like growth factor receptor (Bravo, D. A. et al. (1994) J. Biol. Chem., 269, 25830-25837). The minimal cleavage site is Arg-X-X-Arg′. However, the enzyme prefers the site Arg-X-(Lys/Arg)-Arg′. An additional arginine at the P6 position appears to enhance cleavage (Krysan, D. J. et al. (1999) J. Biol. Chem., 274, 23229-23234). Furin is inhibited by EGTA, α1-Antitrypsin Portland (Jean, F. et al. (1998) Proc. Natl. Acad. Sci. USA, 95, 7293-7298) and polyarginine compounds (Cameron, A. et al. (2000) J. Biol. Chem., 275, 36741-36749).

In some embodiments, the memapsin 2 protein is a protein capable of initiating cleavage of amyloid precursor protein (APP) that contains an amino acid subsequence with at least 80% sequence identity to an amino acid sequence of amino acids 14-501 of FIG. 4. In another embodiment, the memapsin 2 protein contains an amino acid subsequence with at least 90% sequence identity to an amino acid sequence of amino acids 14-501 of FIG. 4. In another embodiment, the memapsin 2 protein that contains an amino acid subsequence with at least 95% sequence identity to an amino acid sequence of amino acids 14-501 of FIG. 4. In another embodiment, the memapsin 2 protein that contains an amino acid subsequence of amino acids 16-456 of FIG. 4 optionally containing at least one conservative amino acid substitution.

In some embodiments, the truncated memapsin 2 protein consists essentially of an amino acid subsequence with at least 80%, at least 90%, or at least 95% sequence identity to an amino acid sequence of amino acids 15-501 of FIG. 4. In other embodiments, the truncated memapsin 2 protein consists essentially of an amino acid subsequence with at least 80%, at least 90%, or at least 95% sequence identity to an amino acid sequence of amino acids 15-454 of FIG. 4. In other embodiments, the truncated memapsin 2 protein consists essentially of an amino acid subsequence of amino acids 15-501 or 15-454 of FIG. 4 optionally containing at least one conservative amino acid substitution. In related embodiments, the truncated memapsin 2 protein consists essentially of an amino acid subsequence of 10 to 486 amino acids. In other related embodiments, the truncated memapsin 2 protein is a protein capable of initiating cleavage of amyloid precursor protein (APP).

In some embodiments, the truncated memapsin 2 protein consists essentially of an amino acid subsequence with at least 80%, at least 90%, or at least 95% sequence identity to an amino acid sequence of amino acids 15-501 of FIG. 4. In other embodiments, the truncated memapsin 2 protein consists essentially of an amino acid subsequence with at least 80%, at least 90%, or at least 95% sequence identity to an amino acid sequence of amino acids 15-454 of FIG. 4. In other embodiments, the truncated memapsin 2 protein consists essentially of an amino acid subsequence of amino acids 15-501 or 15-454 of FIG. 4 optionally containing at least one conservative amino acid substitution. In related embodiments, the amino acid subsequence is 10 to 486 amino acids. In other related embodiments, the truncated memapsin 2 protein is a protein capable of initiating cleavage of amyloid precursor protein (APP).

In some embodiments, the truncated memapsin 2 protein consists essentially of an amino acid subsequence with at least 80%, at least 90%, or at least 95% sequence identity to an amino acid sequence of amino acids 41-454, 43-454, 58-454, or 63-454 of FIG. 4. In other embodiments, the truncated memapsin 2 protein consists essentially of an amino acid subsequence of amino acids 41-454, 43-454, 58-454, or 63-454 of FIG. 4 optionally containing at least one conservative amino acid substitution. In related embodiments, the truncated memapsin 2 protein consists essentially of an amino acid subsequence of 10 to 413 amino acids. In other related embodiments, the truncated memapsin 2 protein is a protein capable of initiating cleavage of amyloid precursor protein (APP).

In some embodiments, the truncated memapsin 2 protein consists essentially of an amino acid sequence with at least 80%, at least 90%, or at least 95% sequence identity to amino acids 41-454, 43-454, 58-454, or 63-454 of FIG. 4. In other embodiments, the truncated memapsin 2 protein consists essentially of amino acids 41-454, 43-454, 58-454, or 63-454 of FIG. 4 optionally containing at least one conservative amino acid substitution.

In some embodiments, the truncated memapsin 2 protein consists of an amino acid sequence with at least 80%, at least 90%, or at least 95% sequence identity to amino acids 41-454, 43-454, 58-454, or 63-454 of FIG. 4. In other embodiments, the truncated memapsin 2 protein consists of amino acids 41-454, 43-454, 58-454, or 63-454 of FIG. 4 optionally containing at least one conservative amino acid substitution.

B. Anti-Truncated Memapsin 2 Antibody

In some embodiments, the present invention provides an anti-truncated memapsin 2 antibody useful in reducing memapsin 2 β-secretase activity in a subject, decreasing levels of β-amyloid peptide in the brain of a subject, treating Alzheimer's disease and/or reducing the size and/or number of β-amyloid plaques in the brain of a subject. An anti-truncated memapsin 2 antibody is an antibody specifically immunoreactive with a truncated memapsin 2 protein, as defined above.

Methods of producing polyclonal and monoclonal antibodies that react specifically with truncated memapsin 2 protein are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Antibodies are purified using techniques well known to those of skill in the art.

A number of truncated memapsin 2 proteins comprising immunogens may be used to produce antibodies specifically reactive with truncated memapsin 2 protein and isotypes thereof. For example, synthetic truncated memapsin 2 protein or a synthetic peptide fragments thereof derived from the sequences disclosed herein, optionally conjugated to a carrier protein can be used as an immunogen. Recombinant protein may be expressed in eukaryotic or prokaryotic cells, and purified using methods known to those of skill in the art. Either monoclonal or polyclonal antibodies may be generated, for subsequent use in immunoassays to measure or isolate the protein.

Anti-truncated memapsin 2 antibodies include chimeric and humanized antibodies, as well as polypeptides having structure and function substantially homologous to antigen-binding sites obtained from such antibodies. Thus, in some embodiments, an anti-truncated memapsin 2 antibody is a peptide defining the CDR region, or a portion of the CDR region sufficient to impart specific immunological reactivity to a truncated memapsin 2 protein.

Monoclonal antibodies and polyclonal sera may be collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against untruncated memapsin 2 protein or other proteins, using a competitive binding immunoassay.

Once truncated memapsin 2 protein specific antibodies are available, truncated memapsin 2 protein can be detected by a variety of immunoassay methods. For a review of immunological and immunoassay procedures, see Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra.

Binding affinity for truncated memapsin 2 protein is typically measured or determined by standard antibody-antigen assays, such as Biacore competitive assays, saturation assays, or immunoassays such as ELISA or RIA.

Such assays can be used to determine the dissociation constant of the antibody. The phrase “dissociation constant” refers to the affinity of an antibody for an antigen. Specificity of binding between an antibody and an antigen may be indicated by reference to the dissociation constant (K_{D}=1/K, where K is the affinity constant=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex). Typically, the binding interactions between antigen and antibody include reversible noncovalent associations such as electrostatic attraction, Van der Waals forces and hydrogen bonds.

In some embodiments, the antibodies of the invention are specifically immunoreactive with truncated memapsin 2 proteins. Where the antibodies are specifically immunoreactive with truncated memapsin 2 proteins, the antibodies may bind to the protein with a K_{D} of less than 0.1 mM, less than 1 μM, less than 0.1 μM, or less than 0.01 μM. Specific polyclonal antisera and monoclonal antibodies may bind with a K_{D} of less than 0.1 mM, less than 1 μM, less than 0.1 μM, or less than 0.01 μM.

Methods of preparing polyclonal antibodies are known to the skilled artisan (e.g., Coligan, supra; and Harlow & Lane, supra). Polyclonal antibodies can be raised in a mammal, e.g., by one or more injections (e.g. subcutaneous or intraperitoneal injections) of a truncated memapsin 2 protein and, if desired, an adjuvant. For example, an inbred strain of mice (e.g., BALB/C mice) or rabbit may be immunized with the protein using an appropriate adjuvant and a standard immunization protocol. The animal's immune response to the immunogen preparation is monitored by taking test bleeds and determining the titer of reactivity to truncated memapsin 2 protein. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

In some embodiments, the truncated memapsin 2 protein is conjugated to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with a truncated memapsin 2 protein to elicit lymphocytes that produce or are capable of producing antibodies that will specifically bind to the immunizing agent. Alternatively, the lymphocytes may be immunized in vitro. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Human antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser, preferably 2, 3, 4, 5, or 6 such sequences. However, it is to be appreciated that some amino acid substitutions within the linker can be made. For example, a valine can be substituted for a glycine.

Methods of making scFv antibodies are generally known. In brief, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

Finding the antibody with the highest affinity is dictated by the efficiency of the selection process and depends on the number of clones that can be screened and the stringency with which it is done. Typically, higher stringency corresponds to more selective panning. If the conditions are too stringent, however, the phage will not bind. After one round of panning, the phage that bind to truncated memapsin 2 proteins or to cells expressing truncated memapsin 2 proteins on their surface are expanded in E. coli and subjected to another round of panning. In this way, an enrichment of many fold occurs in 3 rounds of panning. Thus, even when enrichment in each round is low, multiple rounds of panning will lead to the isolation of rare phage and the genetic material contained within which encodes the scFv with the highest affinity or one which is better expressed on phage.

Regardless of the method of panning chosen, the physical link between genotype and phenotype provided by phage display makes it possible to test every member of a cDNA library for binding to antigen, even with large libraries of clones.

In one embodiment, the antibodies are bispecific antibodies. Bispecific antibodies are monoclonal, preferably human or humanized, antibodies that have binding specificities for at least two different antigens or that have binding specificities for two epitopes on the same antigen.

C. Nucleic Acids

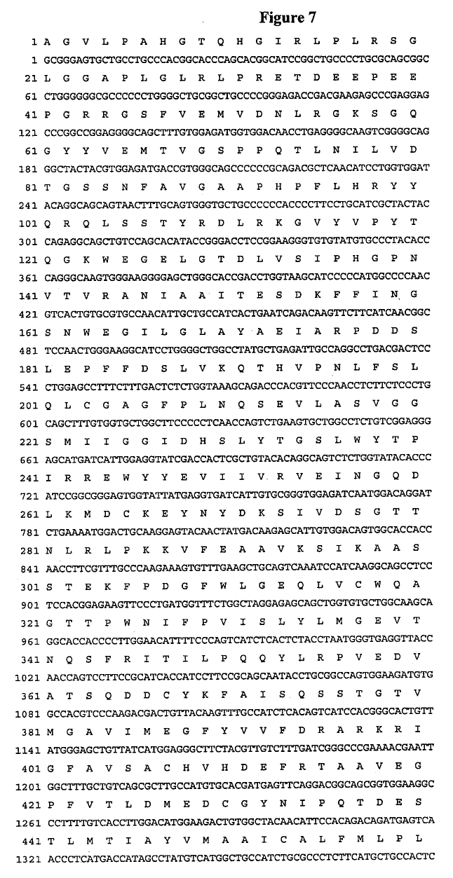

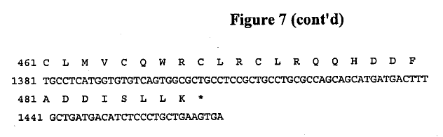

In some embodiments, the present invention provides an isolated nucleic acid encoding a truncated memapsin 2 protein or an anti-truncated memapsin 2 antibody. In some embodiments, where the isolated nucleic acid encodes a truncated memapsin 2 protein, the isolated nucleic acid selectively hybridizes to a nucleic acid sequence of FIG. 7. In other embodiments, the isolated nucleic acid hybridizes under stringent hybridization conditions to a nucleic acid sequence of FIG. 7. In some embodiments the hybridization reaction is incubated at 42° C. in a solution comprising 50% formamide, 5×SSC and 1% SDS, and washed at 65° C. in a solution comprising 0.2×SSC and 0.1% SDS.

In an exemplary embodiment, the isolated nucleic acid contains a subsequence having at least 70% nucleic acid sequence identity to a nucleic acid sequence of FIG. 7. In a related embodiment, the nucleic acid has 75%, 76%, 77%, 78%, 79%, 80%, 85%. 86%, 87%, 88%, 89%, 90%, 91%, 92%, 93%, 94%, 95%, 96%, 97%, or 98% sequence identity. in some related embodiments, the nucleic acid sequence includes a subsequence with sequence identity to any portion of the nucleic acid sequence of FIG. 7 corresponding to the truncated memapsin 2 protein sequences discussed above (e.g. amino acids 41-454, 43-454, 58-454, or 63-454 of FIG. 4).

The present invention also provides expression vectors containing the above nucleic acids and host cells transfected with the vectors.

D. General Recombinant DNA Methods

The production of proteins and antibodies of the current invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression. A Laboratory Manual (1990); and Current Protocols in Molecular Biology (Ausubel et al., eds., 1994)).

For nucleic acids, sizes are given in either kilobases (Kb) or base pairs (bp). These are estimates derived from agarose or acrylamide gel electrophoresis, from sequenced nucleic acids, or from published DNA sequences. For proteins, sizes are given in kilodaltons (kD) or amino acid residue numbers. Protein sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

1. Expression in Prokaryotes and Eukaryotes

To obtain high level expression of a cloned gene, such as those cDNAs encoding proteins and antibodies, one typically subclones the protein or antibody into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing proteins and antibodies are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the protein- or antibody-encoding nucleic acid in host cells. A typical expression cassette thus contains a promoter operably linked to the nucleic acid sequence encoding antibody or peptide and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins and antibodies from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with protein- or antibody-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein or antibody, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein or antibody.

After the expression vector is introduced into the cells, the transfected cells are cultured under conditions favoring expression of the peptide or antibody, which is recovered from the culture using standard techniques identified below.

2. Purification of Proteins and Antibodies

Proteins and antibodies can be purified from any suitable expression system by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra, and Sambrook et al., supra).

Recombinant proteins and antibodies may be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins and antibodies expressed in bacteria may form insoluble aggregates (“inclusion bodies”). Several protocols are suitable for purification of the protein or antibody inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Proteins and antibodies are separated from other bacterial proteins by standard separation techniques, e.g., with Ni-NTA agarose resin.

Alternatively, it is possible to purify the proteins and antibodies from the bacteria periplasm. After lysis of the bacteria, when the proteins and antibodies are exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the proteins and antibodies can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The proteins and antibodies can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### III. Methods

The present invention provides novel methods of reducing memapsin 2 β-secretase activity in a subject, decreasing levels of β-amyloid peptide in the brain of a subject, treating Alzheimer's disease and/or reducing the size and/or number of β-amyloid plaques in the brain of a subject. The methods may include the step of administering an effective amount of truncated memapsin 2 protein, anti-truncated memapsin 2 antibody, and/or nucleic acid encoding a truncated memapsin 2 protein or anti-truncated memapsin 2 antibody.

A “patient” or “subject” for the purposes of the present invention includes both humans and other animals, particularly mammals. Thus the methods are applicable to both human therapy and veterinary applications. In some embodiments, the patient or subject is a mammal, such as a primate. In other embodiments, the patient or subject is human.

It has been discovered that administering an effective amount of a truncated memapsin 2 protein, anti-truncated memapsin 2 antibody, nucleic acid encoding a truncated memapsin 2 protein, or nucleic acid encoding an anti-truncated memapsin 2 antibody to a subject provides surprisingly effective properties as a therapeutic approach to, for example, reducing Aβ production and/or modifying the progression of AD. The administration of truncated memapsin 2 protein antigen to a subject results in the production of antibody specifically immunoreactive with a truncated memapsin 2 protein (also referred to herein as anti-truncated memapsin 2 antibodies). Thus, anti-truncated memapsin 2 antibodies inhibit β-secretase activity, resulting in a reduction of Aβ production. Therefore, the present invention provides truncated memapsin 2 protein antigen, anti-truncated memapsin 2 antibodies, and nucleic acids encoding a truncated memapsin 2 or anti-truncated memapsin 2 antibodies useful in reducing Aβ production, treating the progression of AD, reducing memapsin 2 β-secretase activity, decreasing levels of β-amyloid peptide in the brain of a subject, and/or reducing the size or number of β-amyloid plaques in the brain.

A. Administering Proteins and Antibodies

In some embodiments, the present invention provides a method of reducing memapsin 2 β-secretase activity in a subject, decreasing levels of β-amyloid peptide in the brain of a subject, treating Alzheimer's disease and/or reducing the size and/or number of β-amyloid plaques in the brain of a subject. The method includes the step of administering an effective amount of a truncated memapsin 2 protein (or pharmaceutical composition containing truncated memapsin 2 protein) to a subject in need of such treatment.

In another embodiment, the present invention provides a method of reducing memapsin 2 β-secretase activity in a subject, decreasing levels of 2β-amyloid peptide in the brain of a subject, treating Alzheimer's disease and/or reducing the size and/or number of β-amyloid plaques in the brain of a subject. The method includes the step of administering an effective amount of anti-truncated memapsin 2 antibody (or pharmaceutical composition containing anti-truncated memapsin 2 antibody) to a subject in need of such treatment.

The proteins, antibodies, and pharmaceutical compositions thereof may be administered parenterally, i.e., intraarticularly, intravenously, intraperitoneally, subcutaneously, or intramuscularly, intravenously or intraperitoneally by a bolus injection. For example, see, Stadler, et al., U.S. Pat. No. 5,286,634, which is incorporated herein by reference.

In other methods, the proteins, antibodies, and pharmaceutical compositions thereof may be contacted with the target tissue by direct application of the preparation to the tissue. The application may be made by topical, “open” or “closed” procedures. By “topical”, it is meant the direct application of the pharmaceutical preparation to a tissue exposed to the environment, such as the skin, oropharynx, external auditory canal, and the like. “Open” procedures are those procedures which include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrazamide imaging of the spinal cord. Alternatively, the preparations may be administered through endoscopic devices.

The preparations can also be administered in an aerosol inhaled into the lungs (see, Brigham, et al., Am. J. Sci. 298(4):278-281 (1989)) or by direct injection at the site of disease (Culver, HUMAN GENE THERAPY, MaryAnn Liebert, Inc., Publishers, New York. pp. 70-71 (1994)).

B. Administering Nucleic Acid Encoding a Truncated Memapsin 2

In another embodiment, the present invention provides a method of reducing memapsin 2 β-secretase activity in a subject, decreasing levels of β-amyloid peptide in the brain of a subject, treating Alzheimer's disease and/or reducing the size and/or number of β-amyloid plaques in the brain of a subject. The method includes the step of administering an effective amount of a nucleic acid encoding a truncated memapsin 2 protein (or pharmaceutical composition containing nucleic acid encoding a truncated memapsin 2) to a subject in need of such treatment. Thus, in some embodiments, the present invention provides nucleic acid vaccines, wherein the nucleic acid encodes a truncated memapsin 2 protein.

In general, nucleic acid vaccines are different in structure from traditional vaccines. In some embodiments, the vaccine is encoded by a plasmid, such as small rings of double-stranded DNA originally derived from bacteria, but totally unable to produce an infection.

Nucleic acid vaccines may be delivered to a subject (including mammals such as humans) to induce a therapeutic or prophylactic immune response. The nucleic acid vaccines of the present invention may be delivered by injection, which puts the genes directly into some cells and also leads to uptake by cells in the vicinity of the inserted needle. Once inside cells, some of the recombinant plasmids enter the nucleus and direct synthesis of the encoded antigenic proteins. Those proteins can elicit humoral (antibody-mediated) immunity when they escape from cells, and they can elicit cellular (killer-cell) immunity when they are broken down and properly displayed on the cell surface. The nucleic acid vaccines are easy to design and to generate in large quantities using recombinant DNA technology.

Vaccine delivery vehicles can be delivered in vivo by administration to an individual patient, for example, by systemic administration (e.g., intravenous, intraperitoneal, intramuscular, subdermal, intracranial, anal, vaginal, oral, buccal route or they can be inhaled) or they can be administered by topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

A large number of delivery methods are well known to those of skill in the art. Such methods include, for example liposome-based gene delivery (Debs and Zhu (1993) WO 93/24640; Mannino and Gould-Fogerite (1988) BioTechniques 6(7): 682-691; Rose U.S. Pat No. 5,279,833; Brigham (1991) WO 91/06309; and Felgner et al. (1987) Proc. Natl. Acad. Sci. USA 84: 7413-7414), as well as use of viral vectors (e.g., adenoviral (see, e.g., Berns et al. (1995) Ann. NY Acad. Sci. 772: 95-104; Ali et al. (1994) Gene Ther. 1: 367-384; and Haddada et al. (1995) Curr. Top. Microbiol. Immunol. 199 ( Pt3): 297-306 for review), papillomaviral, retroviral (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin, et al. (1984) Mol. Cell. Biol., 4:2072-2081; Hermonat and Muzyczka (1984) Proc. Natl. Acad. Sci. USA, 81:6466-6470; McLaughlin et al. (1988) and Samulski et al. (1989) J. Virol., 63:03822-3828), and the like.

“Naked” DNA and/or RNA that comprises a nucleic acid vaccine can be introduced directly into a tissue, such as muscle. See, e.g., U.S. Pat. No. 5,580,859. Other methods such as “biolistic” or particle-mediated transformation (see, e.g., Sanford et al., U.S. Pat. No. 4,945,050; U.S. Pat. No. 5,036,006) are also suitable for introduction of nucleic acid vaccines into cells of a mammal according to the invention. These methods are useful not only for in vivo introduction of DNA into a mammal, but also for ex vivo modification of cells for reintroduction into a mammal. As for other methods of delivering nucleic acid vaccines, if necessary, vaccine administration is repeated in order to maintain the desired level of immunomodulation.

Nucleic acid vaccine vectors (e.g., adenoviruses, liposomes, papillomaviruses, retroviruses, etc.) can be administered directly to the mammal for transduction of cells in vivo.

C. Administering Nucleic Acid Encoding Anti-Truncated Memapsin 2 Antibody

In another embodiment, the present invention provides a method of reducing memapsin 2 β-secretase activity in a subject, decreasing levels of β-amyloid peptide in the brain of a subject, treating Alzheimer's disease and/or reducing the size and/or number of β-amyloid plaques in the brain of a subject. The method includes the step of administering an effective amount of a nucleic acid encoding an anti-truncated memapsin 2 antibody (or pharmaceutical composition containing nucleic acid encoding anti-truncated memapsin 2 antibody) to a subject in need of such treatment.

In some embodiments, the nucleic acid encoding anti-truncated memapsin 2 antibody is useful in gene therapy. In this regard, genes encoding the anti-truncated memapsin 2 antibody are introduced into a suitable mammalian host cell for expression or coexpression using a number of viral based systems which have been developed for gene transfer into mammalian cells. For example, retroviruses provide a convenient platform for gene delivery systems. A selected nucleotide sequence encoding a V_{H} and/or a V_{L} domain polypeptide can be inserted into a vector and packaged in retroviral particles using techniques known in the art. The recombinant virus can then be isolated and delivered to a subject. A number of suitable retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109. Some methods for producing and using retroviral vectors for gene therapy herein are described, for example, in International Publication No. WO 91/02805, published Mar. 7, 1991, and in U.S. patent application Ser. No. 08/404,796, filed Mar. 15, 1995 for “Eukarotic Layered Vector Initiation Systems;” Ser. No. 08/405,627, filed Mar. 15, 1995 for “Recombinant .alpha.-Viral Vectors;” and Ser. No. 08/156,789, filed Nov. 23, 1993 for “Packaging Cells.”

A number of suitable adenovirus vectors have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have been developed recently for gene delivery. Such systems can include control sequences, such as promoter and polyadenylation sites, as well as selectable markers or reporter genes, enhancer sequences, and other control elements which allow for the induction of transcription. AAV vectors can be readily constructed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors which will find use for delivering the present nucleic acid molecules encoding the Fab molecules include those derived from the pox family of viruses, including vaccinia virus and avian poxvirus. By way of example, vaccinia virus recombinants expressing the genes can be constructed as follows. The DNA encoding the particular Fab molecule is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the Fab molecule into the viral genome. The resulting Tₖ-recombinant can be selected by culturing the cells in the presence of 5-bromodeoxyuridine and picking viral plaques resistant thereto.

A vaccinia based infection/transfection system can be conveniently used to provide for inducible, transient expression of the Fab molecules in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

Alternatively, avipoxviruses, such as the fowlpox and canarypox viruses, can also be used to deliver the Fab-encoding nucleotide sequences. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., the International Publications WO 91/12882; WO 89/03429, published Apr. 20, 1989; and WO 92/03545, published Mar. 5, 1992.

Molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

### IV. Pharmaceutical and Immunogenic Compositions

A. Antibody Pharmaceutical Formulations

In some embodiments, the antibodies of the invention are formulated in pharmaceutical composition. The exact dose is ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for the age, body weight, general health, sex, diet, time of administration, drug interaction and the severity of the condition may be necessary, and will be ascertainable with routine experimentation by those skilled in the art.

The administration of the antibodies of the present invention can be done in a variety of ways including, but not limited to, orally, subcutaneously, intravenously, intranasally, transdermally, intraperitoneally, intramuscularly, intrapulmonary, vaginally, rectally, or intraocularly.

The pharmaceutical compositions comprise an antibody of the invention in a form suitable for administration to a patient. In some embodiments, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following excipients: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody of the invention dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gillman, The Pharmacological Basis of Therapeutics, supra.

The compositions containing antibodies of the invention can be administered for therapeutic or prophylactic treatments. In therapeutic applications, compositions are administered to a patient suffering from a disease in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of Alzheimer's disease (AD) in a mammal is referred to as a “prophylactically effective dose.” The particular dose required for a prophylactic treatment will depend upon the medical condition and history of the mammal, as well as other factors such as age, weight, gender, administration route, efficiency, etc. Such prophylactic treatments may be used, e.g., in a mammal who has previously had AD to prevent a recurrence of the AD, or in a mammal who is suspected of having a significant likelihood of developing AD.

B. Truncated Memapsin 2 Protein Compositions

In some embodiments, the truncated memapsin 2 protein is administered in a pharmaceutical composition. The characteristics of antibody pharmaceutical compositions discussed above, including dosages, methods of administration, forms, salts, buffers, binders, and carrier proteins, are equally applicable to truncated memapsin 2 protein pharmaceutical compositions.

In some embodiments, the truncated memapsin 2 protein pharmaceutical composition additionally includes a pharmaceutically acceptable adjuvant. Safe adjuvants have been developed in the recent years for producing immune response in human proteins since such responses are useful for immunotherapy of cancers. One of the more successful adjuvant is QS-21 (Antigenics, N.Y.) which has been used to elicit immune responses of cancer antigens (Kensil and Kammer, 1998) and human Aβ peptide (Selkoe and Schenk, 2002). Useful adjuvants include, for example, aluminum adjuvants, calcium phosphate nanoparticles, CpG adjuvants, QS-21 adjuvant, MF-59 adjuvant, ISA-51 adjuvant, ISCOM, and PROVAX.

Aluminum adjuvants include aluminum phosphate (AlPO₄), aluminum hydroxide (Al(OH)₃), and alum precipitated vaccines, historically referred to as protein aluminate. These adjuvants are currently the most commonly used and the only type of adjuvants in FDA approved human vaccines. These adjuvants, also referred to as “alum”, are commercially available in different forms (e.g., Alhydrogel, aluminum hydroxide gel adjuvant manufactured by Superfos Biosector a/s, Vedbæk, Denmark) and can be prepared under GMP conditions into an adjuvant gel.

Calcium phosphate nanotechnology-based vaccine adjuvant, BioVant, has been developed by BioSante Pharmaceuticals, Inc. and is being widely used in clinical trials.

CpG adjuvants may also be used in conjunction with truncated memapsin 2 protein. CpG refers to cytosine- and guanine-rich oligonucleotide motifs found in bacteria but not in human. Thus, such nucleotide sequences stimulate immune responses. Several companies have developed adjuvants consisting of different CpG sequence variations (e.g., VaxImmune is a CpG adjuvant produced by Coley Pharmaceutical Group). CpG adjuvants are being used in clinical trials and may produce predominantly Th1 cytokines as is also true for Freund's adjuvant.

QS-21 adjuvant, produced by Antigenics, is an immune stimulating ingredient derived from the bark of the South American tree Quillaja saponaria Molina, and consists primarily of saponin derivatives. It has been used in over 70 phase 1 & 2 clinical trials and some phase 3 trials. It was used to elicit antibody responses from Aβ immunization in a human clinical trial. Hock et al., Neuron, 38, 547-54 (2003).

MF-59 adjuvant is a water-in-oil based adjuvant that has been used in many vaccine clinical trials against cancers and pathogens. A commercial version, a squalene/water emulsion, is produced by Chiron. MF-59 has frequently been used with MTP-PE (muramyl tripeptide linked covalently with dipalmitoyl phosphatidylethanolamine) in trials.

ISA-51 adjuvant is a montanide adjuvant produced by Seppic, Inc. It has been used in a number of anticancer and antiviral early phase clinical trials.

ISCOM (produced by Iscotec AB, Uppsala, Sweden) is a complex consisting of lipids (cholesterol and phospholipids) and saponins that form complexes with antigens. ISCOM contains cage-like microstructure that appears to enhance immune stimulation.

Other useful commercial adjuvants include PROVAX (IDEC Pharmaceutical), and Syntex Adjuvants (muramyl dipeptide derivative), glutaldehyde crosslinks, conjugation to serum albumin or other carrier proteins (e.g. bacterial proteins).

Adjuvants may be studied in mice for their ability to elicit antibody responses against memapsin 2. For example, several adjuvants may be simultaneously tested in B6 mice. The background strain of Tg2576 (but not the AD mice) may be used in a short (22 week) study in order to select a suitable adjuvant in which the efficacy of the adjuvant will be evaluated thoroughly in long term (12 month) experiments.

In some embodiments, twelve-week old B6; SJL, wild type, mice (140 total) are received during week 1. They are divided into seven groups of 20 mice each. After one week for acclimation, blood is drawn from the saphenous vein and used for background titer levels. Immunization of memapsin 2 with 7 selected adjuvants begins in week 3. Weekly injections are made during week 3 to week 5. Boost 4 is given during week 7. Monthly injections are made beginning in week 11. Blood is collected at weeks 6 and 8, and after week 12, monthly for the duration of the study. Observation of adverse side effects is made throughout. The samples are analyzed for anti-memapsin 2 antibodies. The design of the experiment permits sufficient time to determine the maximum titer for each adjuvant tested. The titer is compared to those from the mice receiving memapsin 2 immunization using a standard adjuvant, such as Freund's adjuvant.

In some embodiments, truncated memapsin 2 protein may be incorporated into an immunogenic composition (e.g., vaccines). Appropriate characteristics of immunogenic compositions and vaccines discussed below in the context of nucleic acids are equally applicable to the truncated memapsin 2 protein immunogenic compositions discussed here. Vaccines can be used to treat or prevent Alzheimer's by eliciting an immune response in a subject. Immunogenic compositions comprise one or more such vaccine compounds and a physiologically acceptable carrier. Vaccines may comprise one or more such compounds and a non-specific immune response enhancer. A non-specific immune response enhancer may be any substance that enhances an immune response to an exogenous antigen. Examples of non-specific immune response enhancers include adjuvants, biodegradable microspheres (e.g., polylactic galactide) and liposomes (into which the compound is incorporated; see, e.g., U.S. Pat. No. 4,235,877). Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Vaccine preparation is generally described in, for example, Powell and Newman, eds., Vaccine Design (the subunit and adjuvant approach), Plenum Press (NY, 1995). Vaccines may be designed to generate antibody immunity and/or cellular immunity. Immunogenic compositions and vaccines within the scope of the present invention may also contain other compounds, which may be biologically active or inactive. For example, one or more immunogenic portions of other antigens may be present, either incorporated into a fusion polypeptide or as a separate compound, within the composition or vaccine. Polypeptides may, but need not, be conjugated to other macromolecules as described. Immunogenic compositions and vaccines may generally be used for prophylactic and therapeutic purposes.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

C. Nucleic Acid Compositions

In some embodiments, the nucleic acid encoding a truncated memapsin 2 protein or anti-truncated memapsin 2 antibody is administered in a pharmaceutical or immunogenic composition. Appropriate characteristics of antibody pharmaceutical compositions discussed above, including dosages, methods of administration, forms, salts, buffers, binders, and carrier proteins, are equally applicable to nucleic acid pharmaceutical compositions. Moreover, appropriate characteristics of immunogenic compositions and vaccines discussed above in the context of truncated memapsin 2 proteins are equally applicable to the nucleic acid immunogenic compositions discussed here.

To maximize the immunotherapeutic effects of DNA vaccines, alternative methods for formulating purified plasmid DNA may be desirable. A variety of methods have been described, and new techniques may become available. Cationic lipids can also be used in the formulation (see, e.g., as described by WO 93/24640; Mannino & Gould-Fogerite, BioTechniques 6(7): 682 (1988); U.S. Pat No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

The administration procedure for DNA is not critical. Vaccine compositions (e.g., compositions containing the DNA expression vectors) can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular patient, and the route of administration.

In therapeutic applications, the vaccines are administered to a patient in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

Suitable quantities of DNA vaccine, e.g., plasmid or naked DNA can be about 1 μg to about 100 mg, preferably 0.1 to 10 mg, but lower levels such as 0.1 to 2 mg or 1-10 μg can be employed. In some embodiments, the dose of a nucleic acid composition is from about 1 μg to 100 μg for a typical 70 kilogram patient. Subcutaneous or intramuscular doses for nucleic acid (typically DNA encoding a fusion protein) may range from 0.1 μg to 500 μg for a 70 kg patient in generally good health. Subcutaneous or intramuscular doses for viral vectors comprising the truncated memapsin 2 protein may range from 105 to 109 pfu for a 70 kg patient in generally good health. For example, naked DNA or polynucleotide in an aqueous carrier, can be injected into tissue, e.g., intramuscularly or intradermally, in amounts of from 10 μl per site to about 1 ml per site. The concentration of polynucleotide in the formulation is from about 0.1 μg/ml to about 20 mg/ml.

The vaccine may be delivered in a physiologically compatible solution such as sterile PBS in a volume of, e.g., one ml. The vaccines may also be lyophilized prior to delivery. As well known to those in the art, the dose may be proportional to weight.

The compositions included in the vaccine regimen can be administered alone, or can be co-administered or sequentially administered with other immunological, antigenic, vaccine, or therapeutic compositions. These include adjuvants (such as those discussed above), and chemical or biological agent given in combination with, or recombinantly fused to, an antigen to enhance immunogenicity of the antigen. Such other compositions can also include purified antigens from the immunodeficiency virus or a second recombinant vectors system that expresses f such antigens and is thus able to produce additional therapeutic compositions. For examples, adjuvant compositions can include expression vectors encoding biological response modifiers. Again, co-administration is performed by taking into consideration such known factors as the age, sex, weight, and condition of the particular patient, and, the route of administration.

The vaccines can additionally be complexed with other components such as peptides, polypeptides and carbohydrates for delivery. For example, expression vectors, i.e., nucleic acid vectors that are not contained within a viral particle, can be complexed to particles or beads that can be administered to an individual, for example, using a vaccine gun.

Nucleic acid vaccines are administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), each of which is incorporated herein by reference. One skilled in the art would know that the choice of a pharmaceutically acceptable carrier, including a physiologically acceptable compound, depends, for example, on the route of administration of the expression vector.

For example, naked DNA or polynucleotide in an aqueous carrier can be injected into tissue, such as muscle, in amounts of from 10 μl per site to about 1 ml per site. The concentration of polynucleotide in the formulation is from about 0.1 μg/ml to about 20 mg/ml.

The nucleic acid vaccines obtained using the methods of the invention can be formulated as pharmaceutical compositions for administration in any suitable manner, including parenteral (e.g., subcutaneous, intramuscular, intradermal, or intravenous), topical, oral, rectal, intranasal, intravaginal, intrathecal, buccal (e.g., sublingual), or local administration, such as by aerosol or transdermally, for prophylactic and/or therapeutic treatment. In such compositions the nucleic acid vector can be in admixture with a suitable carrier, diluent, or excipient such as sterile water, physiological saline, glucose or the like. Pretreatment of skin, for example, by use of hair-removing agents, may be useful in transdermal delivery. Suitable methods of administering such packaged nucleic acids are available and well known to those of skill in the art, and, although more than one route can be used to administer a particular composition, a particular route can often provide a more immediate and more effective reaction than another route.

The expression vectors of use for the invention can be delivered to the interstitial spaces of tissues of a patient (see, e.g., Feigner et al., U.S. Pat. Nos. 5,580,859, and 5,703,055). Administration of expression vectors of the invention to muscle is a particularly effective method of administration, including intradermal and subcutaneous injections and transdermal administration. Transdermal administration, such as by iontophoresis, is also an effective method to deliver expression vectors of the invention to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647).

The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. Nos. 5,846,978, 5,663,169, 5,578,597, 5,502,060, 5,476,874, 5,413,999, 5,308,854, 5,192,668, and 5,187,074.

The vaccines can be incorporated, if desired, into liposomes, microspheres or other polymer matrices (see, e.g., Feigner et al., U.S. Pat. No. 5,703,055; Gregoriadis, Liposome Technology, Vols. I to III (2nd ed. 1993). Liposomes, for example, which consist of phospholipids or other lipids, are nontoxic, physiologically acceptable and metabolizable carriers that are relatively simple to make and administer. Liposomes include emulsions, foams, micelles, insoluble monolayers, liquid crystals, phospholipid dispersions, lamellar layers and the like.

Liposomes for use in the invention may be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

In some embodiments, the nucleic acid vaccine is directly introduced into the cells of the individual receiving the vaccine regimen. This approach is described, for instance, in Wolff et. al., Science 247:1465 (1990) as well as U.S. Pat. Nos. 5,580,859; 5,589,466; 5,804,566; 5,739,118; 5,736,524; 5,679,647; and WO 98/04720. The nucleic acids may also be administered using ballistic delivery as described, for instance, in U.S. Pat. No. 5,204,253 or pressure (see, e.g., U.S. Pat. No. 5,922,687). Using this technique, particles comprised solely of DNA are administered, or in an alternative embodiment, the DNA can be adhered to particles, such as gold particles, for administration.

As is well known in the art, a large number of factors can influence the efficiency of expression of antigen genes and/or the immunogenicity of DNA vaccines. Examples of such factors include the reproducibility of inoculation, construction of the plasmid vector, choice of the promoter used to drive antigen gene expression and stability of the inserted gene in the plasmid.

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins. If required, supercoiled DNA can be isolated from the open circular and linear forms using gel electrophoresis or other methods.

### V. Immunoassays

In another aspect, the present invention includes immunoassays useful in detecting truncated memapsin 2 protein and/or cells expressing truncated memapsin 2 protein using anti-truncated memapsin 2 antibodies. For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the truncated memapsin 2 protein or antigenic subsequence thereof). The antibody (e.g., anti-truncated memapsin 2 antibody) may be produced by any of a number of means well known to those of skill in the art and as described above. For isolation of truncated memapsin 2 protein, typically the antibody/antigen complex is dissociated by washing using means known to those of skill in the art. In such applications, typically the antibody is fixed to a substrate such as a plate or a column via covalent or non-covalent linkages (e.g., streptavidin, protein A, protein G, secondary antibodies, and the like). When the assay is used for monitoring and adjusting the dose of truncated memapsin 2 protein administered to a patient, a standard curve of known concentrations of truncated memapsin 2 protein is prepared, for comparison with test results and for quantitating the amount of truncated memapsin 2 protein in the sample. Typically, the standard curve is generated using the same methodology as is used to detect truncated memapsin 2 protein in the patient sample, e.g., ELISA, immunoprecipitation, and the like. Preferred immunoassays of the invention include western blots, ELISA, immunoprecipitation, in situ immunohistochemistry, and immunofluorescence assays.

In another embodiment, anti-truncated memapsin 2 antibodies are detected using immunoassays such as ELISA assays, where the antibody is captured by truncated memapsin 2 protein or an immunogenic fragment thereof.

Immunoassays also often use a labeling agent to specifically bind to and label the complex formed by the antibody and antigen. The labeling agent may itself be one of the moieties comprising the antibody/antigen complex. Thus, the labeling agent may be a labeled truncated memapsin 2 protein or a labeled anti-truncated memapsin 2 antibody. Alternatively, the labeling agent may be a third moiety, such a secondary antibody, that specifically binds to the antibody/truncated memapsin 2 protein complex (a secondary antibody is typically specific to antibodies of the species from which the first antibody is derived). Other proteins capable of specifically binding immunoglobulin constant regions, such as protein A or protein G, may also be used as the label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Immunoassays for detecting and/or isolating truncated memapsin 2 protein in samples may be either competitive or noncompetitive. Noncompetitive immunoassays are assays in which the amount of antigen is directly measured. In a “sandwich” assay, for example, the anti-truncated memapsin 2 antibody antibodies can be bound directly to a solid substrate on which they are immobilized. These immobilized antibodies then capture truncated memapsin 2 protein present in the test sample. Truncated memapsin 2 protein thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety. Methods of binding molecules to a solid support, either covalently or non-covalently, are well known to those of skill in the art. A variety of solid supports known to those of skill in the art, e.g., plates, columns, dipsticks, membranes, and the like, can be used with the present invention.

In competitive assays, the amount of truncated memapsin 2 protein present in the sample is measured indirectly by measuring the amount of a known, added (exogenous) truncated memapsin 2 protein displaced (competed away) from an anti-truncated memapsin 2 antibody by the unknown truncated memapsin 2 protein present in a sample. In one competitive assay, a known amount of truncated memapsin 2 protein is added to a sample and the sample is then contacted with an antibody that specifically binds to truncated memapsin 2 protein. The amount of exogenous truncated memapsin 2 protein bound to the antibody is inversely proportional to the concentration of truncated memapsin 2 protein present in the sample. In one embodiment, the antibody is immobilized on a solid substrate. The amount of truncated memapsin 2 protein bound to the antibody may be determined either by measuring the amount of truncated memapsin 2 protein present in a truncated memapsin 2 protein/antibody complex, or alternatively by measuring the amount of remaining uncomplexed protein. The amount of truncated memapsin 2 protein may be detected by providing a labeled truncated memapsin 2 protein molecule.

A hapten inhibition assay is another competitive assay. In this assay the known truncated memapsin 2 protein, is immobilized on a solid substrate. A known amount of anti-truncated memapsin 2 antibody is added to the sample, and the sample is then contacted with the immobilized truncated memapsin 2 protein. The amount of anti-truncated memapsin 2 antibody bound to the known immobilized truncated memapsin 2 protein is inversely proportional to the amount of truncated memapsin 2 protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

An antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

Immunoassays in the competitive binding format can also be used for crossreactivity determinations. For example, a polypeptide comprising at least an antigenic subsequence of truncated memapsin 2 protein can be immobilized to a solid support. Proteins (e.g. prepromemapsin 2) are added to the assay that compete for binding of the antisera to the immobilized antigen. The ability of the added proteins to compete for binding of the antisera to the immobilized protein is compared to the ability of truncated memapsin 2 protein to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps a truncated memapsin 2 protein, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the immunogen protein that is required to inhibit 50% of binding, then the second protein is said to specifically bind to the polyclonal antibodies generated to a truncated memapsin 2 protein immunogen.

Western blot (immunoblot) analysis is used to detect and quantify the presence of truncated memapsin 2 protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind truncated memapsin 2 protein. The anti-truncated memapsin 2 antibody antibodies specifically bind to the truncated memapsin 2 protein on the solid support. These antibodies may be directly labeled or, alternatively, may be subsequently detected using labeled antibodies (e.g., labeled sheep anti-mouse antibodies) that specifically bind to the anti-truncated memapsin 2 antibody antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize truncated memapsin 2 protein, or secondary antibodies that recognize anti-truncated memapsin 2 antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### VI. Kits and Other Uses

The present invention also provides kits for detecting and isolating truncated memapsin 2 protein, as well as kits for detecting anti-truncated memapsin 2 antibody antibodies. For example, such kits can comprise any one or more of the following materials: anti-truncated memapsin 2 antibody, reaction tubes, and instructions for detecting truncated memapsin 2 protein. Optionally, for detection of anti-truncated memapsin 2 antibody, the kit contains truncated memapsin 2 protein peptide.

The nucleic acids of the subject invention can be useful as probes to identify complementary sequences within other nucleic acid molecules or genomes. Such use of probes can be applied to identify or distinguish infectious strains of organisms in diagnostic procedures or in Alzheimer's research where identification of particular organisms or strains is needed. As is well known in the art, probes can be made by labeling the nucleic acid sequences of interest according to accepted nucleic acid labeling procedures and techniques.

### VII. Examples

Preparation of recombinant human promemapsin 2—Human promemapsin 2 was prepared according to Lin et al. 2000 and is described in detail in U.S. Pat. No. 6,545,127 and United States Patent Application 20020220079. Briefly, a fragment of human prepromemapsin 2 (FIG. 4) from amino acids 14-454 was cloned into the BamHI site of expression vector pET11a and transfected into E. coli BL21(DE3). This portion of human prepromemapsin 2 included the activation peptide, but excluded the transmembrane domain and cytosolic domain. Expression of promemapsin 2 protein as inclusion bodies was initiated by addition of IPTG to the culture at an O.D. of 0.75 and culture was continued for 3 h. Cells were lysed and the inclusion body protein washed with a triton X- 100 buffer, and then dissolved in 8 M urea buffer. Promemapsin 2 protein was refolded by rapid dilution into a Tris buffer, followed by stepwise adjustment of the pH from 9.0 to 8.0 over 48 h. Refolded promemapsin 2 was separated from misfolded protein by gel filtration chromatography.

Preparation of truncated memapsin 2—Recombinant human promemapsin 2 was truncated to various lengths by different methods, described in Ermolieff et al. 2000 and in US application 20020164760. In one method, attenuation occurred spontaneously during the preparation of promemapsin 2 by prolonged incubation for 2-3 weeks at pH 8 either before or after separation by gel filtration. In another method, promemapsin 2 was activated by incubation with clostripain (Ermolieff et al., 2000). Truncated memapsin 2 preparations were purified by ion exchange FPLC using a 6 ml Resource Q column and eluting with a gradient of 0-0.3 M NaCl over 10 column volumes in a 20 mM TrisHCl, 0.4 M urea buffer, pH 8.0.

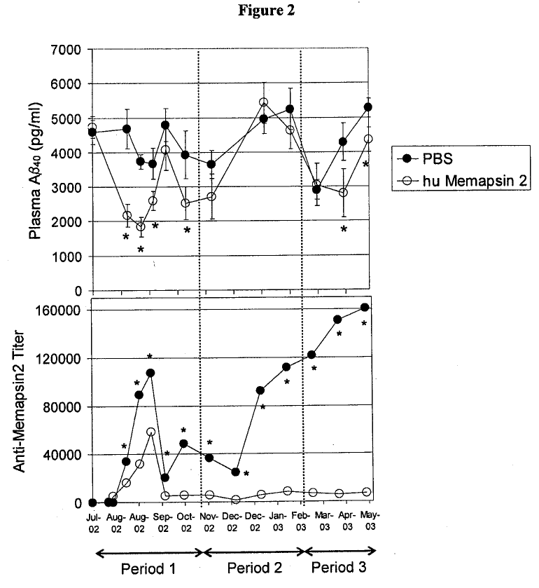

Immunization of transgenic AD mice with full-length promemapsin 2 and truncated memapsin 2 preparations—The first immunization study (FIG. 2) was carried out in three periods. For the first period, ten mice Tg2576 were immunized with spontaneously truncated human memapsin 2 at 6 μg per mouse starting at age of 4 weeks and ten mice were control immunized with only adjuvant and PBS. Eight intradermal immunizations were made over a period of about 5 months. After the fourth immunization, the anti-memapsin 2 antibody titer showed significant elevation in the treatment mice over the controls (FIG. 2). Although some fluctuation of titers was seen, the difference between the two groups maintained throughout this period. During the same period, statistically significant difference between the plasma Aβ₄₀ levels of the experimental and control groups started after the fifth immunization (FIG. 2). his difference, up to about 60% reduction of plasma Aβ₄₀, also persisted during the first five months, albeit with some fluctuation. These results suggest that antibodies to memapsin 2 immunization suppressed plasma Aβ₄₀. Since we had previously demonstrated that there is an efflux of Aβ₄₀ between the brain and plasma (Chang et al., 2003), the difference in plasma Aβ₄₀ must have originated from the difference in brain Aβ₄₀ of the two groups. Also, the plasma Aβ₄₀ and Aβ₄₂ were nearly a constant ratio (Chang et al., 2003), thus the observed Aβ₄₀ reduction was representative of the total Aβ reduction.

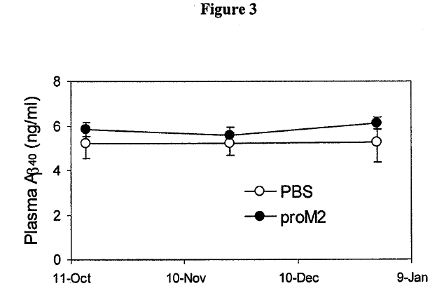

In period two, the possibility of increasing the difference of plasma Aβ₄₀ by an increase of immunogen, promemapsin 2, to 100 μg per mouse we explored. Three immunizations were made over a three-month period. The difference of plasma Aβ₄₀ between the two groups, however, disappeared. Accordingly, a second cohort of animals, initiated by immunization with 100 μg promemapsin 2, revealed no difference in plasma Aβ₄₀ levels between treated and control groups (FIG. 3). These results together indicated that the reduction of Aβ₄₀ was a result of immunization with truncated memapsin 2, and that certain domains of promemapsin 2 may have caused an immune response not conducive to reduction of amyloid beta production.

In period three (FIG. 2), the first cohort of animals (FIG. 2) was immunized with 6 μg of memapsin 2 truncated by activation with clostripain. Three immunizations were made over a three-month period. Again, a statistically significant difference was observed in the plasma Aβ₄₀ between the two groups (FIG. 2).

Sufficient sustained reduction of amyloid peptide in periods 1 and 3, from immunization with truncated memapsin 2, resulted in significant reduction of amyloid burden in the brain. Bielschowski histochemical staining of brain sections from immunized and control animals indicates significant reduction of amyloid burden (Table 1) in regions of the brain typically populated with by amyloid plaque in AD patients. These results indicate that the antibody to memapsin 2, elicited from immunization with truncated memapsin 2, capably crossed the blood-brain barrier to reduce amyloid burden in the brain of the Tg2576 mouse. Antibodies to memapsin 2 may be administered exogenously (passive immunity) to reduce amyloid burden or Aβ levels.

These results established that the immunization of truncated memapsin 2 elicit a response of effective antibody species and resulted in the suppression of Aβ₄₀ production in the brain and plasma.

TABLE 1
Amyloid burden¹ in Tg2576 mice immunized
with truncated memapsin 2

| Brain Region | Control | Treatment | p-value² |
|---|---|---|---|
| Olfactory Bulb | 5.40 | 4.00 | 0.275 |
| Frontal | 9.33 | 11.13 | 0.151 |
| Parietal/Temporal | 20.00 | 7.50 | 0.00004 |
| Hippocampus | 7.18 | 0.88 | 0.001 |
| BG + Th. + Hypo. + CC | 0.45 | 0.00 | 0.026 |
| Brainstem | 0.09 | 0.00 | 0.170 |
| Cerebellum | 0.36 | 0.00 | 0.170 |

¹Bielschowski silver staining
²Student's T-test

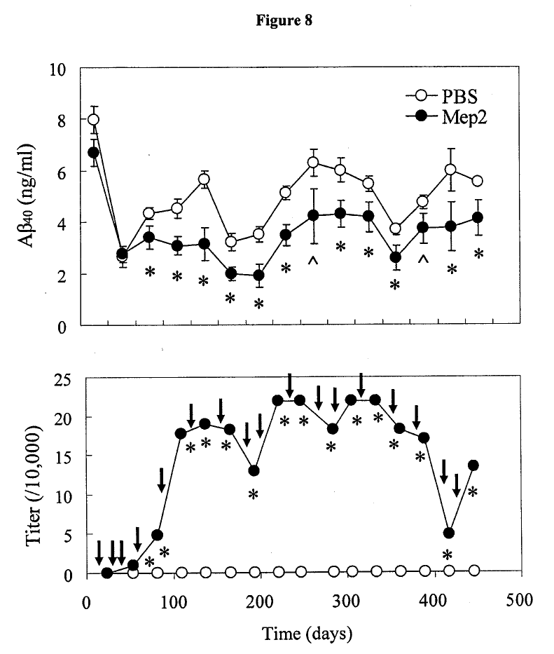

In a separate trial, 4-week-old transgenic AD mice Tg2576 (n=10) were repeatedly immunized with either PBS (control group) or with attenuated memapsin 2 (Mep 2 group) over a 14-month period (arrows in FIG. 8, lower panel). The initial immunization was done by emulsifying either PBS or attenuated memapsin 2 with complete Freund's adjuvant. For the three subsequent immunizations, either PBS or attenuated memapsin 2 was emulsified with incomplete Freund's adjuvant. All other immunizations in the weeks following used either PBS or attenuated memapsin 2 without adjuvant. Animals were subcutaneously injected with 100 μl of either PBS or with 6 μg of attenuated memapsin 2 in 100 μl of 0.4 M urea, 20 mM Tris-HCl, pH 8.0, with or without adjuvant as described. Blood samples were taken from the saphenous vein and plasma separated by centrifugation. Concentrations of plasma Aβ₄₀ were determined by sandwich ELISA. A reduction in plasma Aβ₄₀ was found for the attenuated memapsin 2-immunized group as compared to the control group (FIG. 8, upper panel). This reduction, which started with the rise of anti-memapsin 2 titer at about week 3 (FIG. 8, lower panel), ranged from about 30% to 50% of total and was statistically significant at nearly all time points.

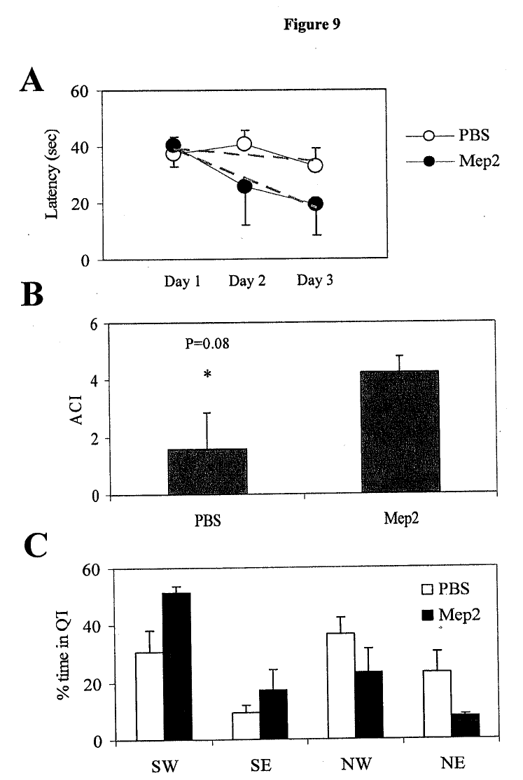

At about 15 months of age, mice were subjected to behavioral testing for cognition in the Morris water maze. Mice from both the control (PBS) and attenuated memapsin 2 groups (Mep 2, FIG. 9A) were analyzed for latency in time to locate a hidden platform submerged in opaque water, using spatial cues to test for reference memory. A significant improvement in the reference memory training was seen in the attenuated memapsin 2-immunized animals relative to the control group (FIG. 9A; p=0.012, difference between slopes). On day 3 of the trial, the platform was removed to assess the retention of spatial memory in a probe trial. The ratio of the number of times crossing the location of the absent platform in the correct quadrant relative to the average number of virtual crossings in the other three quadrants (annulus crossing index, or ACI) is expected to be lower for mice that do not retain spatial memory. Indeed, the mice immunized with attenuated memapsin 2 (FIG. 9B) demonstrated a greater ACI than those in the control group (p=0.08). Additionally, the percentage of time spent in the probe (SW) quadrant was significantly higher for the mice immunized with attenuated memapsin 2 (p=0.02), with a concomitant significant reduction relative to control animals for occupancy of the opposite (NE) quadrant (FIG. 9C; p=0.04).

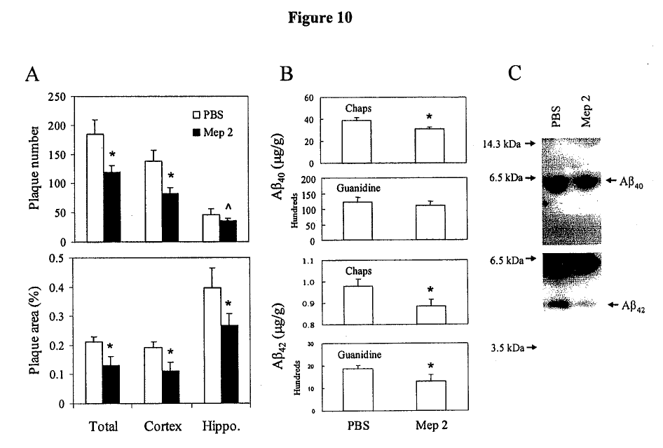

Brains of the animals in the study were collected for analysis of brain Aβ₄₀ and Aβ₄₂, and for histologic determination of the number and area of brain amyloid plaques by Bielschowski or amyloid staining (FIG. 10). It was found that both total plaque numbers and plaque area in immunized mice were reduced (FIG. 10A) by about 35% (p=0.012 and 0.017, respectively). In the attenuated memapsin 2-immunized mice, the plaque areas were lower in the cortex (p=0.019) and hippocampus (p=0.056). The plaque number was reduced in the cortex (p=0.006). However, despite the significant reduction of plaque area in the hippocampus, the reduction of plaque number in the hippocampus was not statistically significant (p=0.15), which appears to be a result of inhibition of plaque expansion rather than nucleation in the hippocampus of attenuated-memapsin 2-immunized animals (FIG. 10A). Using ELISA, Aβ₄₀ and Aβ₄₂ were assessed in the extracts from one of the two brain hemispheres. In the attenuated memapsin 2-immunized mice, Aβ₄₂ was significantly lower (ranging from 30% to 50%, FIG. 10B) in both the CHAPS-buffer extract (soluble aggregates of Aβ) (p=0.0029) and the guanidine-buffer extract (insoluble Aβ deposit) (p=0.037). The lowering of Aβ₄₀ (about 20%), which was less than that for Aβ₄₂, was significant in CHAPS-buffer extract (p=0075). Immunoblotting of Aβ peptides from characteristic samples confirmed the larger difference in Aβ₄₂ than in Aβ₄₀ (FIG. 10C). Statistical analysis supports the contention that differences in plasma Aβ₄₀ and brain Aβ₄₀ and Aβ₄₂ existed between the control and immunized groups.

In conclusion, immunization of AD mice with attenuated memapsin 2 results in a 35 to 40% reduction in the level of Aβ in the plasma, with about the same amount of reduction in brain plaque load and the amount of Aβ peptide in the brain. The reduction of Aβ₄₂ in the brain is particularly significant. This observed reduction of Aβ was accompanied by an improvement in both reference memory training and retention of spatial memory in the Morris water maze, representative of a disease-modifying therapy for Alzheimer's disease.

1. A method of reducing memapsin 2 β-secretase activity to treat a disease associated with memapsin 2 β-secretase activity in a subject in need of such treatment, the method comprising administering to said subject an effective amount of a truncated memapsin 2 protein, and wherein said truncated memapsin 2 protein consists of amino acids 63-454 of FIG. 4, optionally containing at least one conservative amino acid substitution.

2. The method of claim 1, wherein said subject is a mammal.

3. The method of claim 2, wherein said subject is a human.

4. The method of claim 1, wherein said disease is Alzheimer's disease.

5-9. (canceled)

10. The method of claim 1, wherein said truncated memapsin 2 protein consists of amino acids or 63-454 of FIG. 4.

11. A method of decreasing levels of β-amyloid peptide in the brain of a subject, said method comprising administering to said subject an effective amount of a truncated memapsin 2 protein, and wherein said truncated memapsin 2 protein consists of amino acids 63-454 of FIG. 4, optionally containing at least one conservative amino acid substitution, and said truncated memapsin 2 protein reduces memapsin 2 β-secretase activity in said subject.

12. A method of reducing the size or number of β-amyloid plaques in the brain of said subject, said method comprising administering to said subject an effective amount of a truncated memapsin 2 protein, and wherein said truncated memapsin 2 protein consists of amino acids 63-454 of FIG. 4, optionally containing at least one conservative amino acid substitution, and said truncated memapsin 2 protein reduces memapsin 2 β-secretase activity in said subject.

13. A method of treating Alzheimer's disease in a patient in need of such treatment, said method comprising administering to said subject an effective amount of a truncated memapsin 2 protein, and wherein said truncated memapsin 2 protein consists of amino acids 63-454 of FIG. 4, optionally containing at least one conservative amino acid substitution, and said truncated memapsin 2 protein reduces memapsin 2 β-secretase activity in said subject.

14. An antibody specifically immunoreactive with a truncated memapsin 2 protein, and wherein said truncated memapsin 2 protein consists of amino acids 63-454 of FIG. 4, optionally containing at least one conservative amino acid substitution, and said antibody reduces memapsin 2 β-secretase activity.

15. The antibody of claim 14, wherein said antibody is an isolated antibody.

16. The antibody of claim 14, wherein said antibody is a monoclonal antibody.

17. The antibody of claim 14, wherein said antibody is an antibody fragment.

18. The antibody of claim 14, wherein said antibody is a humanized antibody.

19. A pharmaceutical composition comprising the antibody of claim 14 and a pharmaceutically acceptable excipient.

20. A pharmaceutical composition comprising a truncated memapsin 2 and a pharmaceutically acceptable adjuvant, wherein said truncated memapsin 2 protein consists of amino acids 63-454 of FIG. 4, optionally containing at least one conservative amino acid substitution.

21. A pharmaceutical composition comprising a nucleic acid encoding a truncated memapsin 2 and a pharmaceutically acceptable excipient, wherein said truncated memapsin 2 protein consists of amino acids 63-454 of FIG. 4, optionally containing at least one conservative amino acid substitution.

22. An antibody specifically immunoreactive with a truncated memapsin 2 protein, and wherein said truncated memapsin 2 protein consists of amino acids 63-454 of FIG. 4, optionally containing at least one conservative amino acid substitution, and said antibody reduces Aβ production.
